(19) United States
(12) Patent Application Publication
(10) Pub. No.: US 2024/0041007 A1
(43) Pub. Date: Feb. 8, 2024

(54) BRACE FOR PET

(71) Applicant: Proxihealthcare Inc., Ulsan (KR)

(72) Inventor: Young Wook Kim, Seoul (KR)

(21) Appl. No.: 18/377,295

(22) Filed: Oct. 5, 2023

Related U.S. Application Data
(63) Continuation of application No. PCT/KR21/19079, filed on Dec. 15, 2021.

(30) Foreign Application Priority Data
Apr. 8, 2021 (KR) 10-2021-0046130

(51) Int. Cl. A01K 27/00; A61D 9/00

(52) U.S. Cl. CPC A01K 27/009; A61D 9/00; A61N 1/36021

(57) ABSTRACT

A brace for a pet to be worn on body parts of the pet, the brace includes: a protective cover configured to cover at least some of the body parts of the pet; an electrode unit disposed on an inner side surface of the protective cover and configured to receive a driving signal to provide electromagnetic waves corresponding to the driving signal; and a power supply device disposed on an outer side surface of the protective cover and configured to supply the driving signal through an output terminal thereof.

### CROSS REFERENCE TO RELATED APPLICATIONS

This application is a Bypass Continuation of International Application No. PCT/KR2021/019079, filed on Dec. 15, 2021, and claims priority from and the benefit of Korean Patent Application No. 10-2021-0046130, filed on Apr. 8, 2021, each of which is incorporated by reference for all purposes as if fully set forth herein.

### BACKGROUND

### Field

The present invention relates to a brace for pets using special microcurrent electromagnetic waves.

### Discussion of the Background

Pets, such as dogs and cats, experience abnormalities in parts of their body such as spines and kneecaps due to genetic factors, acquired living conditions, or external chocks. To alleviate the symptoms of these abnormalities, devices (e.g., spinal braces, kneecap braces, and so on) have recently been used that are applied in the form of wraps around the affected body parts.

However, with conventional pet braces, therapeutic effects such as pain relief are not be expected, and there are problems with skin problems caused by skin waste when the pet braces are worn.

The above information disclosed in this Background section is only for understanding of the background of the inventive concepts, and, therefore, it may contain information that does not constitute prior art.

### SUMMARY

An object of the present invention for solving the above-mentioned problems is to provide a brace for pets that protects abnormal areas of the pet's body while expecting therapeutic effects on the abnormal areas by providing electromagnetic waves.

Another object of the present invention is to provide a brace for pets that may effectively prevent skin problems that may occur when the brace is worn by using a driving signal generated by mixing AC and DC signals.

Additional features of the inventive concepts will be set forth in the description which follows, and in part will be apparent from the description, or may be learned by practice of the inventive concepts.

According to one aspect of the invention, a brace for a pet is a brace to be worn on body parts of the pet, which includes: a protective cover configured to cover at least some of the body parts of the pet; an electrode unit disposed on an inner side surface of the protective cover and configured to receive a driving signal to provide electromagnetic waves corresponding to the driving signal; and a power supply device disposed on an outer side surface of the protective cover and configured to supply the driving signal through an output terminal thereof.

The electrode unit may include: a first electrode terminal and a second electrode terminal installed while passing through the protective cover; a first electrode connected to one side of the first electrode terminal; and a second electrode connected to one side of the second electrode terminal.

The output terminal may include: a first output terminal and a second output terminal respectively coupled to the other side of the first electrode terminal and the other side of the second electrode terminal exposed to the outside of the protective cover.

A first protrusion may be formed on one of the other side of the first electrode terminal and the first output terminal, and a first engagement groove into which the first protrusion is fitted may be formed on the other of the other side of the first electrode terminal and the first output terminal; and a second protrusion may be formed on one of the other side of the second electrode terminal and the second output terminal, and a second engagement groove into which the second protrusion is fitted may be formed on the other of the other side of the second electrode terminal and the second output terminal.

The brace for a pet may further include: an electrode cover disposed on an upper side of the electrode unit and having a plurality of apertures.

The brace may further include: at least one fixing band extending from the protective cover for a fixed installation of the protective cover.

The driving signal may be generated by mixing an AC signal and a DC signal.

The power supply device may include: a battery; a DC-DC converter configured to receive a voltage from the battery and convert it to an output voltage; a signal generator configured to generate the AC signal using the output voltage of the DC-DC converter; a filter configured to perform a filtering operation on the AC signal generated by the signal generator; and a calibrator configured to generate the driving signal by mixing the AC signal supplied through the filter with the DC signal.

The power supply device may further include: a voltage divider configured to divide the output voltage of the DC-DC converter to generate the DC signal.

The power supply device may further include: a first indicator configured to indicate a state of charge of the battery; and a second indicator configured to indicate a period of time during which the brace is being operated.

According to the present invention as described above, it is possible a brace for pets that protects abnormal areas of the pet's body while expecting therapeutic effects on the abnormal areas by providing electromagnetic waves.

In addition, according to the present invention, it is possible to provide a brace for pets that may effectively prevent skin problems that may occur when the brace is worn by using a driving signal generated by mixing AC and DC signals.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 is a diagram illustrating a brace for pets according to an embodiment of the present invention.

FIG. 2 is a diagram illustrating an outer side surface of a protective cover according to an embodiment of the present invention.

FIG. 3 is a diagram illustrating an inner side surface and an electrode unit of a protective cover according to an embodiment of the present invention.

FIG. 4 is a diagram illustrating an electrode cover according to an embodiment of the present invention.

FIGS. 5A and 5B are diagrams illustrating the power supply device according to an embodiment of the present invention.

FIGS. 6A and 6B are diagrams explaining a biofilm removal effect by a driving signal generated by mixing an AC signal and a DC signal.

FIG. 7 is a diagram illustrating an internal configuration of a power supply device according to an embodiment of the present invention.

FIGS. 8A, 8B, and 8C are diagrams illustrating the waveform of a signal according to an embodiment of the present invention.

FIG. 9 is a diagram illustrating an internal configuration of a power supply device according to another embodiment of the present invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

In this specification, the distinction between elements is only a distinction by a main function performed by each element. That is, two or more elements to be described below may be combined into one element, or one element may be divided into two or more elements according to subdivided functions. Also, each element to be described below may further perform some or all of functions performed by other elements in addition to the main function thereof, and a part of the main function of each element may be performed by other elements.

Hereinafter, a brace for pets according to embodiments of the present invention will be described with reference to the drawings related to the embodiments.

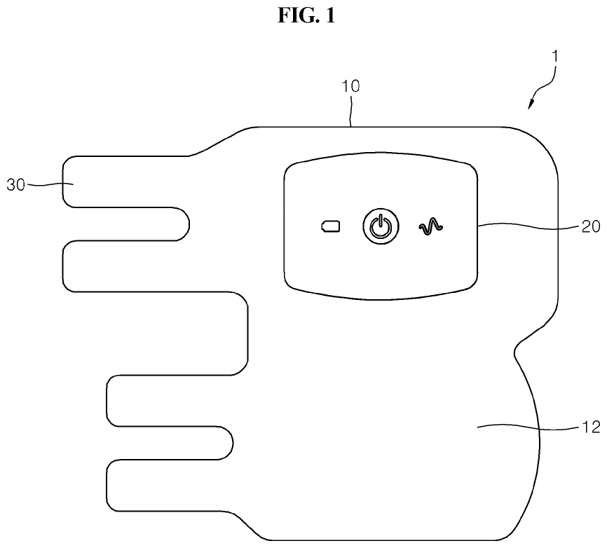

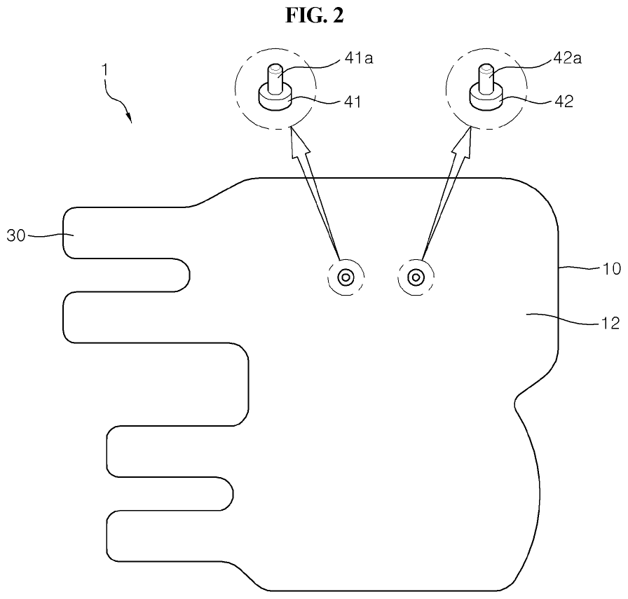

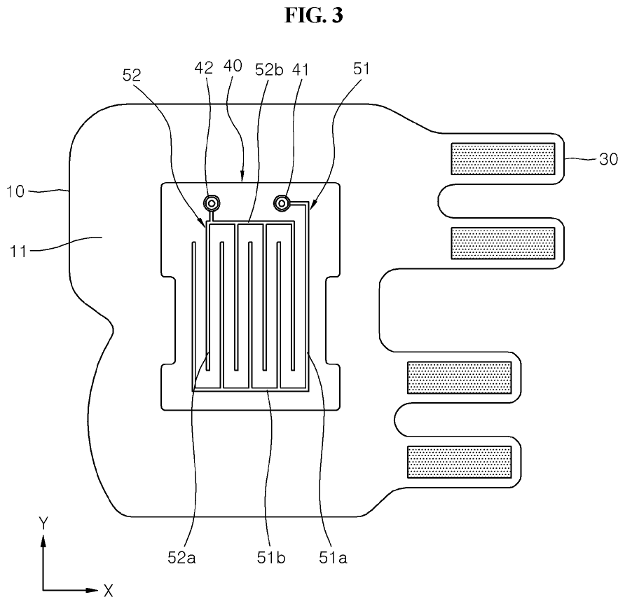

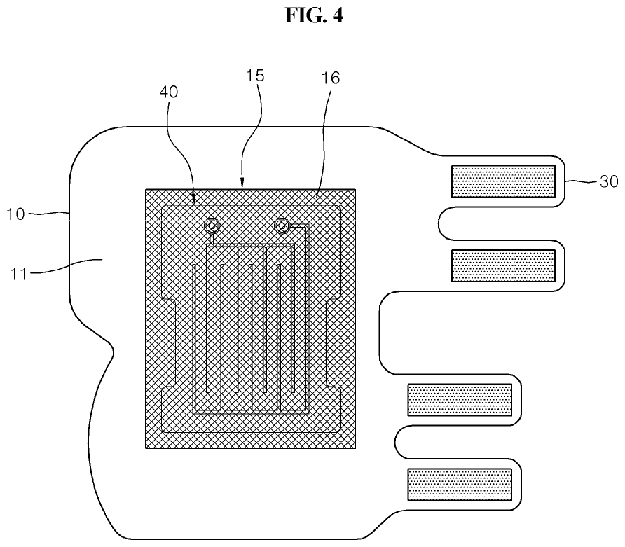

FIG. 1 is a diagram illustrating a brace for pets according to an embodiment of the present invention, FIG. 2 is a diagram illustrating an outer side surface of a protective cover according to an embodiment of the present invention, FIG. 3 is a diagram illustrating an inner side surface and an electrode unit of a protective cover according to an embodiment of the present invention, and FIG. 4 is a diagram illustrating an electrode cover according to an embodiment of the present invention.

A pet brace 1 according to an embodiment of the present invention may be worn on body parts of a pet, thereby performing functions such as protection and pain relief for the body parts.

Referring to FIGS. 1 and 3, the pet brace 1 may include a protective cover 10, an electrode unit 40, and a power supply device 20.

The protective cover 10 may have a size of a certain area and cover at least some of the body parts of the pet when the pet brace 1 is worn, and may be formed of flexible material. For example, the protective cover 10 may be formed of, but is not limited to, fabrics, fiber materials, etc.

In addition, when the pet is wearing the pet brace 1, the inner side surface 11 of the protective cover 10 may be defined as one side surface in contact with the wearing area, and the outer side surface 12 of the protective cover 10 may be defined as the other side surface opposite to the inner side surface 11.

For a fixed installation of the protective cover, at least one fixing band 30 may be formed to extend from the protective cover 10. In an example, the fixing band 30 may be formed to extend from one side of the protective cover 10 to wrap around a body part of the pet and be attached to the other side of the protective cover 10, or it may be interconnected with a separate protective band extending from the other side of the protective cover 10.

The number, length, formed position, etc. of the fixing bands 30 may vary depending on the purpose (spine protection, kneecap protection, etc.) and shape of the pet brace 1.

The electrode unit 40 may be disposed on the inner side surface 11 of the protective cover 10 and receive a driving signal from the power supply device 20 to generate an electric field corresponding to the driving signal.

The electrode unit 40 may provide electromagnetic waves based on the electrical energy of the driving signal, and these electromagnetic waves have an electrical effect on the body part wearing the pet brace 1, thereby providing effects such as pain relief and inflammation reduction.

The power supply device 20 may be disposed on the outer side surface 12 of the protective cover 10 and supply the driving signal via an output terminal thereof.

In addition, the power supply device 20 may be configured to be removably attached to and detached from the protective cover 10. This may facilitate charging, repairing, and the like of the power supply device 20, and allow the pet brace 1 to be used with the power supply device 20 removed when the supply of electromagnetic waves is not required.

Referring to FIGS. 2 and 3, the electrode unit 40 according to an embodiment of the present invention may include a first electrode terminal 41, a second electrode terminal 42, a first electrode 51, and a second electrode 52.

The first electrode terminal 41 and the second electrode terminal 42 may be positioned at a certain distance apart from each other, and may be installed in such a manner as to pass through the protective cover 10.

Accordingly, one side of the first electrode terminal 41 and one side of the second electrode terminal 42 may be positioned on the inner side surface 11 of the protective cover 10, and the other side of the first electrode terminal 41 and the other side of the second electrode terminal 42 may be positioned on the outer side surface 12 of the protective cover 10.

The first electrode 51 may be connected to one side of the first electrode terminal 41, and the second electrode 52 may be connected to one side of the second electrode terminal 42.

When the pet brace 1 is driven, the first electrode 51 and the second electrode 52 may be set as a positive electrode and a negative electrode, respectively.

In this regard, the first electrode 51 and the second electrode 52 may be formed of a material such as, but not limited to, copper, brass, aluminum, conductive polymer, conductive silicon, or stainless steel, and any materials having conductivity may be used as electrode materials.

For example, the first electrode 51 and the second electrode 52 may have separate insulating layers disposed on the top, bottom, or one side thereof. Further, the first electrode 51 and the second electrode 52 may be formed in the form of flexible printed circuits board (FPCB), but are not limited thereto.

To increase the electric field density, the first electrode 51 and the second electrode 52 may be arranged alternately along a specific direction (e.g., an X-axis direction).

For example, the first electrode 51 may include a plurality of first sub-electrodes 51a arranged along a first direction (e.g., the X-axis direction), and a first connecting electrode 51b connected between the first sub-electrodes 51a and the first electrode terminal 41. In this case, the first sub-electrodes 51a may have a shape extending along a second direction (e.g., a Y-axis direction) that intersects the first direction (e.g., the X-axis direction), and the first connecting electrodes 51b may be connected to each end of the first sub-electrodes 51a.

Further, the second electrode 52 may be include a plurality of second sub-electrodes 52a arranged along the first direction (e.g., the X-axis direction), each second sub-electrode being positioned between the first sub-electrodes 51a, and a second connecting electrode 52b connected between the second sub-electrodes 52a and the second electrode terminal 42. In this case, the second sub-electrodes 52a have a shape extending along the second direction (e.g., the Y-axis direction) that intersects the first direction (e.g., the X-axis direction), and the second connecting electrode 52b may be connected to each end of the second sub-electrode 52a.

However, the structure designed for the first electrode 51 and the second electrode 52 is not limited to thereto and may be changed to various forms.

Referring to FIG. 4, the pet brace 1 according to an embodiment of the present invention may additionally include an electrode cover 15 disposed on the upper side of the electrode unit 40. Such electrode cover 15 may have a mesh-like shape including a plurality of apertures 16, in order to minimize the interference phenomenon of electromagnetic waves provided by the electrode unit 40.

Furthermore, the electrode cover 15 may serve to accommodate the electrode unit therein such that the electrode unit does not escape to the outside, and for this purpose, the electrode cover 15 has a larger area than the electrode unit 40 and has an edge fixedly coupled to the inner side surface 11 of the protective cover 10, thereby forming a receiving space in which the electrode unit 40 is placed.

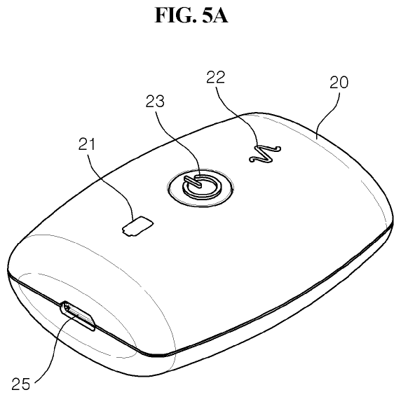

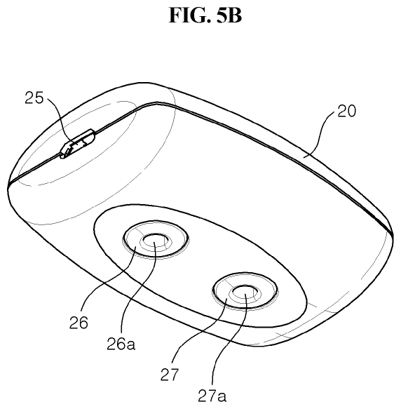

FIGS. 5A and 5B are diagrams illustrating the power supply device according to an embodiment of the present invention.

Referring to FIGS. 5A and 5B, the power supply device 20 according to an embodiment of the present invention may include a first indicator 21, a second indicator 22, and a charging terminal 25.

The first indicator 21 may indicate the charging state of a battery disposed within the power supply device 20. For example, the first indicator 21 may be provided with light-emitting diodes (LED) capable of emitting light in a plurality of colors and may indicate the state of charge of the battery (e.g., red) and a fully charged state of the battery (e.g., blue), and the like.

The second indicator 22 may indicate a period of time during which the pet brace 1 is being operated. For example, the second indicator 22 may be provided with LEDs capable of emitting light in a plurality of colors and may indicate 15 minutes of operating time (e.g., with green), 30 minutes of operating time (e.g., with yellow), and so on.

A switch 23 may be used to control the power ON/OFF and set the operating time of the pet brace 1.

The charging terminal 25 may be used to charge the battery disposed within the power supply device 20.

Meanwhile, the power supply device 20 may be provided with output terminals 26 and 27 for supplying a driving signal and for coupling with the protective cover 10.

A first output terminal 26 may be coupled to the other side of the first electrode terminal 41 exposed to the outside of the protective cover 10, and a second output terminal 27 may be coupled to the other side of the second electrode terminal 42 exposed to the outside of the protective cover 10.

For this purpose, a first protrusion may be formed on one of the other side of the first electrode terminal 41 and the first output terminal 26, and a first engagement groove into which the first protrusion is fitted may be formed on the other of the other side of the first electrode terminal 41 and the first output terminal 26. Further, a second protrusion may be formed on one of the other side of the second electrode terminal 42 and the second output terminal 27, and a second engagement groove into which the second protrusion is fitted may be formed on the other of the other side of the second electrode terminal 42 and the second output terminal 27.

For example, as shown in FIG. 2, a first protrusion 41a and a second protrusion 42a may be formed on the other side of the first electrode terminal 41 and the other side of the second electrode terminal 42, respectively, and as shown in FIG. 5b, a first engagement groove 26a into which the first protrusion 41a is fitted and a second engagement groove 27a in which the second protrusion 42a is fitted may be formed on the first output terminal 26 and the second output terminal 27, respectively.

Conversely, although not separately shown, a first engagement groove and a second engagement groove may be formed on the other side of the first electrode terminal 41 and the other side of the second electrode terminal 42, respectively, and a first protrusion and a second protrusion which are fitted into the first engagement groove and the second engagement groove may be formed on the first output terminal 26 and the second output terminal 27, respectively.

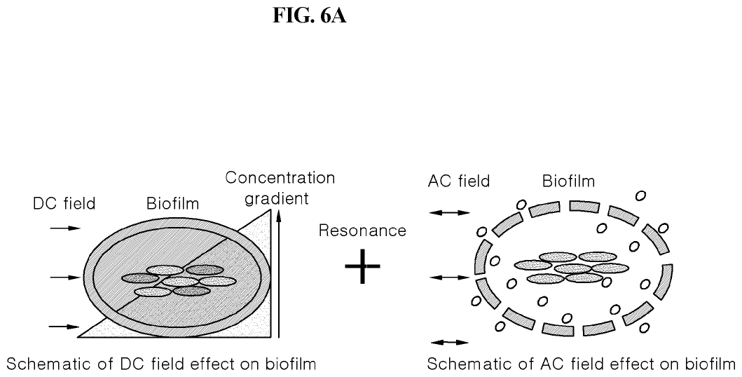

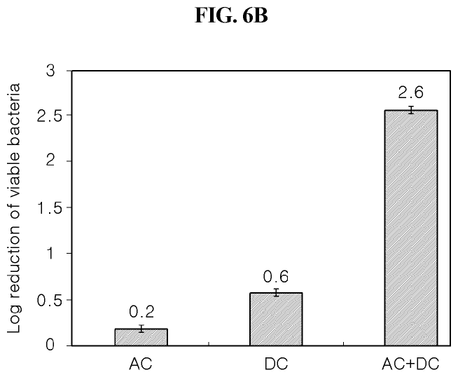

FIGS. 6A and 6B are diagrams explaining a biofilm removal effect by a driving signal generated by mixing an AC signal and a DC signal.

The power supply device 20 may generate the driving signal using a battery voltage supplied from an internal battery, output the generated driving signal to the output terminals 26 and 27, which is then supplied to the electrodes 51 and 52 via the electrode terminals 41 and 42.

In addition, the power supply device 400 may generate the driving signal by mixing an alternating current (AC) signal and a direct current (DC) signal.

Accordingly, the driving signal includes both AC and DC components, and synergistic effects and resonance may occur due to the simultaneous application of the mixture of the AC and DC components, thereby enhancing the removal effect of pore contaminants.

Typical pore contaminants may include biofilms caused by bacteria, and referring to FIG. 6a, an electric field induced by the DC component of the driving signal may increase the structural stress of the biofilm by inducing an imbalance in the local distribution of charges, and an electric field induced by the AC component of the driving signal may increase the permeability into the outer protective substance by generating specific vibrations.

The synergistic effect between these AC and DC components may be observed in FIG. 6B. Specifically, compared to the biofilm removal effect when the electric fields induced by the AC component and the DC voltage are provided separately, it can be seen that the biofilm removal effect is significantly superior when the electric fields induced by the AC component and the DC voltage are provided simultaneously in an overlapping manner.

Since the electric field induced by the DC component and the electric field induced by the AC component from the driving signal supplied from the signal supply apparatus according to an embodiment of the present invention may be provided simultaneously by the electrode unit 40, an enhanced removal effect in relation to debris (i.e., biofilms) in the skin of a pet may be achieved. Consequently, skin problems that may occur when wearing the pet brace 1 may be effectively prevented.

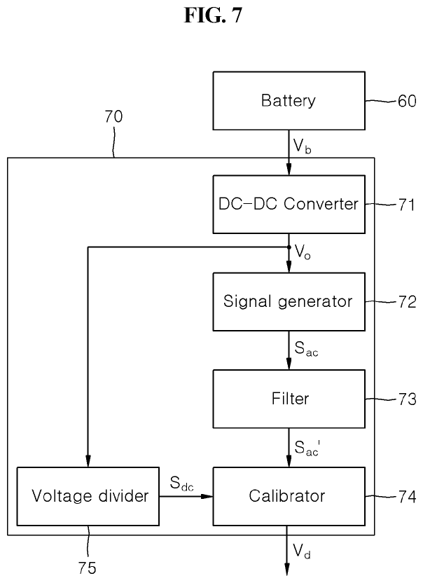

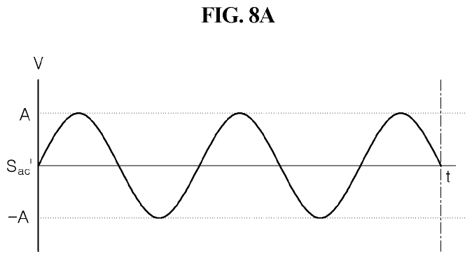

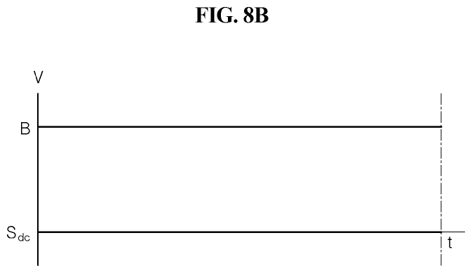

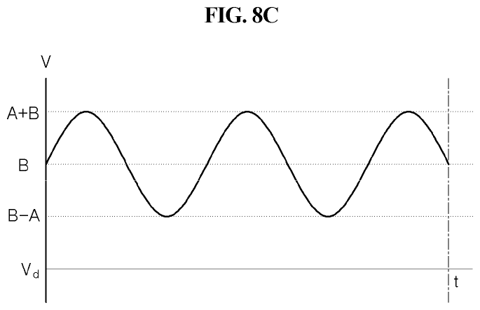

FIG. 7 is a diagram illustrating an internal configuration of a power supply device according to an embodiment of the present invention, and FIGS. 8A, 8B, and 8C are diagrams illustrating the waveform of a signal according to an embodiment of the present invention. In particular, FIG. 8A shows a filtered AC signal Sac′, FIG. 8B shows a DC signal Sdc, and FIG. 8C shows a driving signal Vd generated by mixing the filtered AC signal Sac′ and the DC signal Sdc.

Referring to FIG. 7, the power supply device 20 may include a battery 60 and a signal supply module 70.

The battery 60 may be accommodated inside the power supply device 20 and may provide a battery voltage Vb to the signal supply module 70.

For example, the battery 60 may be set as a primary battery or secondary battery.

When the battery 60 is a primary battery, the user may periodically replace the battery 60, whereas when the battery 60 is a secondary battery, it may be charged via the charging terminal 25 discussed earlier.

The signal supply module 70 may include a DC-DC converter 71, a signal generator 72, a filter 73, and a calibrator 74, and may additionally include a voltage divider 75.

The DC-DC converter 71 may receive the battery voltage Vb from the battery 60, and may convert the battery voltage Vb into a predetermined level of output voltage Vo to output it.

The signal generator 72 may operate based on the voltage supplied from the DC-DC converter 71, and may generate an AC signal Sac with a predetermined frequency using the output voltage Vo from the DC-DC converter 71.

The signal generator 72 may be implemented using a known configuration, such as an oscillator, function generator, or the like, capable of generating an AC signal.

For example, the AC signal Sac may be set to a frequency of 1 KHz to 1000 MHz. This is because if the AC signal Sac is set to a low frequency of less than 1 KHz, the effect of removing pore contaminants (biofilms) is reduced, and if the AC signal Sac is set to an ultra-high frequency of more than 1000 MHz, the effect of removing pore contaminants (biofilms) is also reduced. Meanwhile, the AC signal Sac may be set to a frequency of 5 MHz to MHz, which is suitable for the removal of pore contaminants (biofilms).

Additionally, the amplitude of the AC signal Sac may be set to 0.1 mV to 3V. This is because if the amplitude of the AC signal Sac is less than 0.1 mV, it is difficult to expect a removal effect of the pore contaminants (biofilms), and if the amplitude of the AC signal Sac exceeds 3 V, there is a risk of generating toxic substances due to electrolysis of body fluids.

The filter 73 may perform a filtering operation on the AC signal Sac generated by the signal generator 72. For example, the filter 73 may include a low pass filter and may convert the AC signal Sac in a sawtooth wave form into the AC signal Sac′ in a sine wave form. However, the type of the filter 73 is not limited thereto, and various types of filters may be employed depending on the design structure.

The calibrator 74 may generate the driving signal Vd by mixing the DC signal Sdc with the AC signal Sac′ supplied through the filter 73. For example, the calibrator 74 may be implemented as an operating amplifier capable of summing (or superimposing) the AC signal Sac′ and the DC signal Sdc, but is not limited thereto.

Accordingly, an offset corresponding to the DC signal Sdc may be generated in the AC signal Sac′, and the driving signal Vd containing both AC and DC components may be generated.

Since the driving signal Vd contains all the characteristics of the AC signal Sac, the driving signal Vd may be set to a frequency of 1 KHz to 1000 MHz, and further, may be set to a frequency of 5 MHz to 15 MHz, which is more suitable for the removal of the pore contaminants (biofilms). Additionally, the amplitude of the driving signal Vd may be set to 0.1 mV to 3V.

Referring to FIG. 8A, the calibrator 440 may receive the AC signal Sac′ having an amplitude of A volt (V) from the filter 73, and superimpose the DC signal Sdc of B volt (V) as shown in FIG. 8B onto the AC signal Sac′, thereby generating a final driving signal Vd as shown in FIG. 8C.

In this case, the voltage value of the DC signal Sdc may be set equal to or greater than the amplitude of the AC signal Sac′. Accordingly, the voltage value of the driving signal Vd may be set to 0 or more.

Ultimately, the DC offset value of the driving signal Vd may be set to be equal to or greater than the amplitude of the driving signal Vd.

If the DC offset value of the driving signal Vd is less than the amplitude value of the driving signal Vd, it will result in an interval where the voltage of the driving signal Vd has a negative value, and since the voltage in the interval has a negative value in that segment, a loss of electrical energy occurs.

However, when the DC offset value of the driving signal Vd is set equal to or greater than the amplitude of the driving signal Vd as in the embodiment of the present invention, it is possible to minimize the loss of electrical energy because the voltage of the driving signal Vd is always zero or greater.

Meanwhile, the DC signal Sdc may be generated by the voltage divider 75. For example, the voltage divider 75 may receive the output voltage Vo of the DC-DC converter 71, and perform voltage division on the output voltage Vo to generate the DC signal Sdc. The voltage divider 75 may be composed of a resistor string for dividing the output voltage Vo, but is not limited thereto.

When the output voltage Vo of the DC-DC converter 71 is suitable to be used directly to generate the driving signal Vd, the corresponding output voltage Vo may serve as the DC signal Sdc. In this case, the voltage divider 75 may be omitted, and the output voltage Vo of the DC-DC converter 71 may be inputted to the calibrator 74.

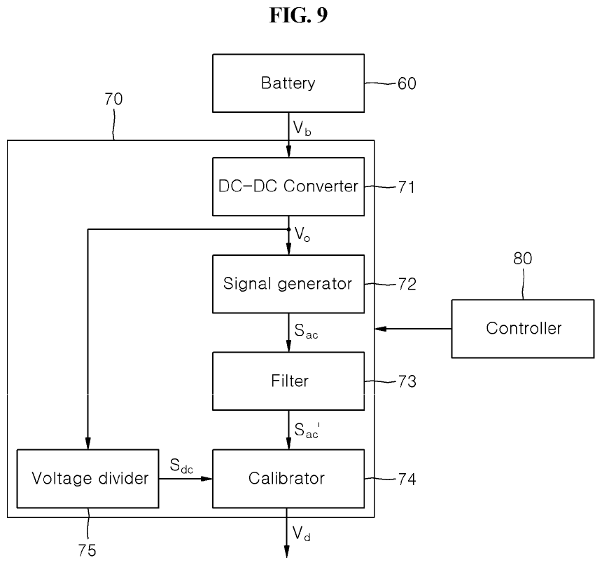

FIG. 9 is a diagram illustrating an internal configuration of a power supply device according to another embodiment of the present invention.

Referring to FIG. 9, in a power supply device 20 according to another embodiment of the present invention, the signal supply module 70 may change at least one characteristic of the driving signal Vd under the control of the user. In addition, the power supply device 20 may additionally include a controller 80 that controls the signal supply module 70 in response to the user's input.

For example, the characteristics of the driving signal Vd may include the amplitude and the DC offset of the driving signal Vd.

Accordingly, by adjusting at least one of the amplitude and DC offset of the driving signal Vd, the user may set an optimal driving signal Vd suitable for the pet. This function enables skin care for pets considering their characteristics.

In this case, the user's input method for controlling the characteristics of the driving signal Vd may be through a switch or a separate button (not shown) installed on the power supply device 20, and the user may adjust or set the characteristics of the driving signal Vd by manipulating the switch 23 or the like installed on the power supply device 20.

When setting information for the driving signal Vd is inputted through button or switch manipulation by the user, the controller 80 may control the signal supply module 70 to provide the driving signal Vd having amplitude and DC offset values corresponding to the inputted setting information.

The controller 80 may change the amplitude of the AC signal Sac by controlling the signal generator 72. Further, the controller 80 may adjust the voltage value of the DC signal Sdc by controlling the DC-DC converter 71 and/or the voltage divider 75. Accordingly, the characteristics of the driving signal Vd may ultimately be changed.

In this case, the controller 80 may control the voltage divider 75 to set the voltage value of the DC signal Sdc to be equal to or greater than the amplitude of the AC signal Sac′. Consequently, the voltage value of the driving signal Vd may be set to be equal to or greater than zero.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A brace for a pet to be worn on body parts of the pet, comprising:
a protective cover configured to cover at least some of the body parts of the pet;
an electrode unit disposed on an inner side surface of the protective cover and configured to receive a driving signal to provide electromagnetic waves corresponding to the driving signal; and
a power supply device disposed on an outer side surface of the protective cover and configured to supply the driving signal through an output terminal thereof.

2. The brace for a pet of claim 1, wherein the electrode unit includes:
a first electrode terminal and a second electrode terminal installed while passing through the protective cover;
a first electrode connected to one side of the first electrode terminal; and
a second electrode connected to one side of the second electrode terminal.

3. The brace for a pet of claim 2, wherein the output terminal includes:
a first output terminal and a second output terminal respectively coupled to the other side of the first electrode terminal and the other side of the second electrode terminal exposed to the outside of the protective cover.

4. The brace for a pet of claim 3, wherein a first protrusion is formed on one of the other side of the first electrode terminal and the first output terminal, and a first engagement groove into which the first protrusion is fitted is formed on the other of the other side of the first electrode terminal and the first output terminal; and
a second protrusion is formed on one of the other side of the second electrode terminal and the second output terminal, and a second engagement groove into which the second protrusion is fitted is formed on the other of the other side of the second electrode terminal and the second output terminal.

5. The brace for a pet of claim 4, further comprising:
an electrode cover disposed on an upper side of the electrode unit and having a plurality of apertures.

6. The brace for a pet of claim 1, further comprising:
at least one fixing band extending from the protective cover for a fixed installation of the protective cover.

7. The brace for a pet of claim 1, wherein the driving signal is generated by mixing an AC signal and a DC signal.

8. The brace for a pet of claim 7, wherein the power supply device includes:
a battery;
a DC-DC converter configured to receive a voltage from the battery and convert it to an output voltage;
a signal generator configured to generate the AC signal using the output voltage of the DC-DC converter;
a filter configured to perform a filtering operation on the AC signal generated by the signal generator; and
a calibrator configured to generate the driving signal by mixing the AC signal supplied through the filter with the DC signal.

9. The brace for a pet of claim 8, wherein the power supply device further includes:
a voltage divider configured to divide the output voltage of the DC-DC converter to generate the DC signal.

10. The brace for a pet of claim 8, wherein the power supply device further includes:
a first indicator configured to indicate a state of charge of the battery; and
a second indicator configured to indicate a period of time during which the brace is being operated.
